(19) United States
(12) Patent Application Publication
(10) Pub. No.: US 2007/0093399 A1
(43) Pub. Date: Apr. 26, 2007

(54) Cleaning, sanitization and regeneration of chromatography media using chlorine dioxide

(75) Inventors: Richard A. Hamilton, Beverly, MA (US); Gary A. O'Neill, Tyngsborough, MA (US)

Correspondence Address: LAHIVE & COCKFIELD, LLP, ONE POST OFFICE SQUARE, BOSTON, MA, 02109-2127, US

(73) Assignee: SELECTIVE MICRO TECHNOLOGIES, LLC, Beverly, MA (US)

(21) Appl. No.: 11/260,672

(22) Filed: Oct. 26, 2005

(51) Int. Cl. A61K 8/00

(52) U.S. Cl. 510/137

(57) ABSTRACT

Disclosed are methods for using chlorine dioxide, e.g., pure chlorine dioxide solutions, for cleaning, regenerating and/or sterilizing chromatographic media. Such methods may be carried out in situ, e.g., without removing the media from a column.

### FIELD OF THE INVENTION

The invention relates generally to methods for cleaning, sanitizing, and/or regenerating chromatography media, e.g., with a chlorine dioxide solution.

### BACKGROUND OF THE INVENTION

The use of gas for retarding, controlling, killing or preventing microbiological contamination (e.g., bacteria, fungi, viruses, mold spores, algae and protozoa); retarding, preventing, or controlling biochemical decomposition; controlling respiration, deodorizing and/or retarding and preventing chemotaxis to name a few, is known. Such gases include, but are not limited to, chlorine dioxide (ClO₂), sulfur dioxide, nitrogen dioxide, nitric oxide, nitrous oxide, carbon dioxide, hydrogen sulfide, hydrocyanic acid, and dichlorine monoxide. For example, the use and efficacy of chlorine dioxide is documented and discussed in various publications such as G. D. Simpson et al., A Focus on Chlorine Dioxide, An Ideal Biocide (visited Feb. 5, 2000) http://clo2.com/readings/waste/corrosion.html and K. K. Krause, DDS et al., The Effectiveness of Chlorine Dioxide in the Barrier System (visited Feb. 5, 2000) http://www.dentallozic.com/dentist/effects.htm.

In particular, chlorine dioxide has been found to be useful as a disinfectant, antiseptic and sanitizer. It is used, e.g., to disinfect drinking water and various water supplies and food items. In addition, chlorine dioxide finds use as a bleaching agent for flour, fats and textiles. Chlorine dioxide also has shown great utility as an antiseptic for treating metal and plastic surfaces, as well as other substrates such as countertops, meat processing and packaging equipment, and dental and medical instruments and devices.

### SUMMARY OF THE INVENTION

The present invention is based, at least in part, on the discovery that chlorine dioxide, e.g., ClO₂ gas in solution, is also effective at cleaning, sanitizing and/or regenerating chromatography media. Accordingly, in some aspects, the present invention is directed to methods for at least partially cleaning, sanitizing, and/or regenerating chromatography media. These methods generally include contacting chromatography media with chlorine dioxide. In some aspects, the present invention provides methods for at least partially regenerating, cleaning and/or sanitizing chromatography media in situ, which methods include contacting chromatography media with chlorine dioxide without removing the chromatography media from its vessel.

In some embodiments, the chlorine dioxide is a substantially pure chlorine dioxide solution. The chlorine dioxide solution can be at least about a 5 ppm chlorine dioxide solution, at least about a 25 ppm chlorine dioxide solution, at least about a 100 ppm chlorine dioxide solution, at least about a 500 ppm chlorine dioxide solution, or at least about a 1000 ppm chlorine dioxide solution. The chlorine dioxide solution can be a buffered aqueous solution or an unbuffered aqueous solution.

In some embodiments, the chromatography media includes, but is not limited to an ion exchange resin, an affinity resin, a Protein A resin, a diatomaceous earth, a silica gel, a silica, a silica-based support system, agarose, sepharose, controlled pore gas and size exclusion media. In some embodiments, the chromatography media has been exposed to cation exchange (CEX) or anion exchange (AEX) proteins or both. In some embodiments, the pH of the chlorine dioxide in solution is between about 3.0 and about 10.0. In some embodiments, the pH of the solution is such that the chlorine dioxide remains stable and intact. In some embodiments, the pH of the solution has little or no effect on the ability of the chlorine dioxide to regenerate, clean or sanitize the chromatography media. In some embodiments, the chromatography media is contacted in a single wash with the chlorine dioxide. In other embodiments, the chromatography media is contacted in two or more washes with the chlorine dioxide. In yet other embodiments, the method is carried out at about room temperature.

In one embodiment, the time needed to regenerate the chromatography media is about 25% less than conventional cleaning, sanitization, or regeneration methods. In some embodiments the time needed to clean, sanitize or regenerate the chromatography media is about 60 minutes, about 30 minutes, about 15 minutes, or about 5 minutes.

In one embodiment, the method allows the effective lifetime of the chromatography media to be increased by at least 20% in comparison to conventional regeneration, cleaning or sanitization methods. In some embodiments, the method allows the chromatography media to be used at least twice, at least five times or at least twenty times.

In one embodiment, the method is carried out without removing the chromatography media from its vessel. In one embodiment, the method is carried out in situ.

In some embodiments, the chlorine dioxide does not have an adverse effect on the function of the chromatography media. In some embodiments, the chromatography media parameters are substantially unaffected by the chlorine dioxide. In other embodiments, any affinity ligands on the chromatography media remain intact during the method. In still other embodiments, the functional capacity of the chromatography media is increased during at least a portion of the method. In yet other embodiments, the chlorine dioxide does not chlorinate the chromatography media.

The method can further include removing any residual chlorine dioxide from the chromatography media. For example, the method can further include removing residual chlorine dioxide under vacuum. In other embodiments, the method can further include flushing with 1 to 2 volumes of water or buffer. In yet other embodiments, the method can further include storing the chromatography media in the chlorine dioxide. In some embodiments, the chlorine dioxide is generated at the point of use.

In some embodiments, the chlorine dioxide can remediate microbiological contaminants. The microbiological contaminants can be, but are not limited to fungi, bacteria, viruses, protista, mildew, molds, mold spores, Tuburculosis, Coronavirus, HIV, Hepatitis A, Rotovirus, Feline calici, Poliovirus, Pseudomonas aeruginosa, Staphylococcus aureus, Salmonella choleraesuis, Enterococcus faecalis, Klebsiella pneumoniae, E. coli, Salmonella typhimurium, T mentag, Penicillin digitatum, Stachybotrys Chartarum, Aspergillus niger, Absidia sp., Acrodontium salmoneum, Aspergillus candidus, smallpox, legionella and/or anthrax. In some embodiments, the amount of colony forming units subsequent to contacting the chromatography media with the chlorine dioxide is less than about 10. In other embodiments, there is a greater than 99% reduction in viable cell count subsequent to contacting the chromatography media with the chlorine dioxide. In yet other embodiments, microbes do not build up a resistance to the chlorine dioxide. In still other embodiments, the chlorine dioxide destroys any biofilm matrix. In some embodiments, the chromatography media is sterilized.

In some embodiments, the methods further include contacting the chromatography media to at least one gas, e.g., N₂, O₂, sulfur dioxide, nitrogen dioxide, nitric oxide, nitrous oxide, carbon dioxide, hydrogen sulfide, hydrocyanic acid and/or dichlorine monoxide.

In some aspects, the present invention includes a kit comprising chlorine dioxide and instructions for using the chlorine dioxide to regenerate, clean, or sanitize chromatography media. The kit may be used in any of the methods presented herein.

### DETAILED DESCRIPTION OF THE INVENTION

It has been discovered that chlorine dioxide (ClO₂), e.g., in pure and substantially pure solutions of chlorine dioxide, can be used effectively to clean, sanitize and/or regenerate many types of chromatography media, including, but not limited to specific chromatography media which can not be cleaned, sanitized and/or regenerated effectively or at all by conventional cleaning methods. Without wishing to be bound by any particular theory, it is believed that chlorine dioxide, e.g., pure and substantially pure solutions of chlorine dioxide at various concentrations, is compatible with many chromatography media, including some media that are incompatible with other cleaning materials. Without wishing to be bound by any particular theory, it is also believed that chlorine dioxide does not interfere with many linking/binding chemistries or functional groups (e.g., affinity ligands). As an example, while many ion exchange resins or size exclusion chromatography media may be robust enough to be cleaned using salt or 1.5 molar caustic solutions, affinity resins or silica-based support system media generally are not. However, these latter media can be effectively cleaned, sanitized and/or regenerated using chlorine dioxide. The present invention is based, at least in part, on the unexpected fact that chlorine dioxide, although a strong oxidizer, is compatible with chromatography media, e.g., affinity or silica-based media.

The present invention is generally directed to methods for at least partially cleaning, sanitizing, and/or regenerating chromatography media. These methods include contacting chromatography media with chlorine dioxide. In some aspects, the present invention provides methods for at least partially regenerating, cleaning and/or sanitizing chromatography media in situ, which methods include contacting chromatography media with chlorine dioxide without removing the chromatography media from its vessel. ClO₂ can be generated or produced from reactants in a variety of forms such as tablets, powders (e.g., zeolites impregnated or otherwise incorporating reactants), liquids (e.g., liquid solutions of reactants), gels, gases, and devices such as those described in U.S. Pat. No. 6,602,466, “Apparatus and Method for Controlled Delivery of a Gas” by Hamilton et al. and International Patent Application Publication No. WO01/60750, same title. The contents of these applications are hereby incorporated in their entireties by this reference. Accordingly, in some embodiments, chlorine dioxide generated or produced from reactants is incorporated into a solution, e.g., a buffered or unbuffered aqueous solution. In some embodiments, the resultant chlorine dioxide solution is a pure chlorine dioxide solution.

In order to more clearly and concisely describe the subject matter of the claims, the following definitions are intended to provide guidance as to the meaning of specific terms used in the following written description, examples and appended claims.

As used herein, the term “chlorine dioxide” is meant to encompass all forms of chlorine dioxide, e.g., pure chlorine dioxide and/or chlorine dioxide in solution, e.g., a buffered or unbuffered aqueous solution. As used herein the term chlorine dioxide “in solution” refers generally to gaseous chlorine dioxide in solution. Unlike many gasses, e.g., chlorine gas, which hydrolyze in water, chlorine dioxide in water does not hydrolyze to any appreciable extent but remains in solution as a dissolved gas. It is approximately 10 times more soluble than chlorine and can be readily removed from dilute aqueous solutions.

The term “chromatography” generally refers to the separation of the components of a mixture. For example, separation can result from differential retardation caused by unequal degrees of sorption of substances washed along in a stream of, e.g., fluid. Additionally or alternatively, separation can result from differential affinities of substances for a gas or liquid mobile medium and for a stationary adsorbing medium through which they pass, e.g., different distributions of solutes as they flow around or over a stationary phase. Separation by chromatography results from partitioning the components of a mixture between the stationary phase and a mobile phase. Solutes held preferentially in the stationary phase are retained longer in the system than those distributed selectively in the mobile phase, so they elute from the system as local concentrations in the mobile phase in the order of increasing distribution coefficients with respect to the stationary phase.

As used herein, the term “chromatography media” refers to material that is capable of being used in chromatography processes. Chromatography media includes, but is not limited to, ion-exchange resins, affinity resins, Protein A resins, hydrophobic interaction media, diatomaceous earth, various silica and silica gels and chromatography papers. In some embodiments, the chromatography media is at least partially spent. As used herein, “spent chromatography media” refers to chromatography media which has been used in one or more separation procedures. In manufacture or through use, chromatography media can become contaminated or lose efficacy if not cleaned, rid of organic matter and/or rid of microbial contamination (sanitized or sterilized).

The present invention may be useful in treating chromatography media from many types of chromatographic processes, e.g., liquid chromatography. Liquid chromatography (LC) is the term used to describe the separation of compounds which are in the liquid phase. Liquid chromatography includes, but is not limited to, separation methods such as adsorption chromatography, partition chromatography, normal-phase LC, reverse-phase LC (RPLC), hydrophobic-interaction chromatography (HIC), size-exclusion chromatography, high-pressure LC (HPLC), thin-layer chromatography (TLC), ion-exchange chromatography (IEC). Other methods of chromatography are known in the art.

As used herein the terms “conventional cleaning methods,” “conventional sanitization methods,” and “conventional regeneration methods” refer to methods to clean, sanitize and/or regenerate chromatography media employed in the art. For example, methods of cleaning, sanitizing or regenerating chromatography media include the use of salt solutions (NaCl), caustic solutions up to 1.5 molar NaOH, acetic acid, detergents, ethanol, isopropanol, acetonitrile and Tris EDTA. While one or more of these materials may be effective to clean, sanitize or regenerate some chromatography media, there can be problems with efficacy, compatibility, residuals, time required or ease of use with these materials or with the chromatography media when some or any of these materials are used to clean the media. Some examples of these problems include: (1) In cleaning precipitated proteins from hydrophobic media, high salt, commonly used with ion exchange media, may cause irreversible binding of precipitated proteins. (2) Lipid removal may be accomplished using organic solvents such as ethanol, isopropanol and acetonitrile. However, in the case of organic solvents, it may be necessary to have explosion-proof facilities solely for a cleaning step, thereby adding an increased cost to production. (3) Endotoxins adhere to anion exchangers and protein affinity media. Sodium hydroxide has been shown to be effective in deactivating endotoxins in some anion exchange media, but cannot be used at effective concentrations on some protein affinity media. There exist other examples.

As used herein, the term “affinity ligand” refers to a moiety which is attached to a stationary phase of a chromatography media. The ligand generally undergoes some type of interaction with a molecule of interest, e.g., a covalent or ionic bond or other structural or non-structural interaction, e.g., neutral recognition bonding or hydrophobic interaction, such that a molecule of interest remains within the column while the rest of the mixture continues through to the end of the column, generally to be removed. Subsequent to this separation, a different chemical can be flushed through the column to detach the molecule of interest from the affinity ligand. In some cases, however, in conventional separation techniques, the molecule of interest remains bound to the affinity ligand. In some embodiments of the present invention, the interaction of the affinity ligand with the molecule of interest is broken, thus liberating a molecule which would, under conventional chromatographic conditions, not be accessible.

As used herein, the term “chromatography media parameters” include those properties that are typically used to characterize substrates for suitability in specific end use applications, such as the separation and purification of biomolecules using size exclusion chromatography, gel filtration chromatography, gel permeation chromatography, hydrophobic interaction chromatography or reversed phase chromatography. Typical chromatography media performance parameters of interest to the chromatography practitioner include, for example, compressibility, permeability (flow resistance), % polymer pore volume (% porosity of polymer bed), pore volume, interparticle void volume (% interstitial volume or excluded volume), % polymer solids (volume), polymer porosity (volume pores/volume polymer).

The term “substantially unaffected,” as used herein, refers generally to the physical and/or chemical properties of the chromatography media which make it suitable for use as a separation media remaining unaffected by the exposure to chlorine dioxide or only being affected to an extent which does not significantly affect its performance as a separation media.

For the purposes of the present invention, here and throughout, the terms “contacting,” “delivering,” and “administering” shall include, but are not limited to methods of washing, soaking, immersing, dipping, bathing, spraying, flushing and storing the chromatography media in the ClO₂. These procedures provide a means for cleaning, sanitizing and/or regenerating the chromatography media of the present invention and all of the methods of the present invention can utilize any of these means and their variants. Cleaning is a general term that includes removal of unwanted chemicals, ions and/or other objects or moieties that may interfere with the performance of chromatography media, as well as providing various levels of biocidal and antimicrobial activity. That is, any and all of the cleaning methods of the present invention can further mean, e.g., deodorizing, sanitizing, disinfecting, sterilizing, and removing and/or preventing biofilm growth or accumulation. Disinfecting generally involves killing pathogenic organisms in water, air, or on surfaces. The term cleaning is meant to include at least partially cleaning as well as substantially cleaning, e.g., removing at least 1%, 5%, 10%, 25%, 50% . . . 100% of unwanted chemicals, ions, etc. As used herein, the term “sterilization” generally involves the removal or destruction of all microorganisms, including pathogenic and other bacteria, vegetative forms, and spores, and includes any one of a number of terms for microbial decontamination or inactivation including sanitization or disinfection. The term sterilization is also meant to encompass the destruction of one or more or all target organisms. That is, in the chromatography art, a specific known organism may be retained on the column. In some embodiments, it is one or more of these specific organisms that is targeted in sterilization. The term “sanitizing,” as used herein, is meant to include at least partially reducing the number of microorganisms as well as substantially reducing the number of microorganisms, e.g., removing and/or destroying at least 1×10, 1×10², 1×10³, 1×10⁴, 1×10⁵, 1×10⁶ microorganisms. In some embodiments, sanitization includes sterilization, e.g., where all microorganisms or all desired microorganisms are destroyed. When chromatography media is used in separations where the end product will be ingested or injected into living organisms, microbial decontamination and cleaning is useful. As used herein, the term “regeneration” includes the restoration of separation efficacy to the media. Regeneration can further increase the effectiveness of the media or extend its useful life. The term regenerating is meant to include at least partially regenerating as well as substantially regenerating, e.g., restoring at least 1%, 5%, 10%, 25%, 50% . . . 100% of separation efficacy to the chromatography media. All values in between the values listed above in relation to cleaning, sanitizing and regenerating are meant to be encompassed by the present invention, e.g., destroying 58% of microorganisms and/or restoring 31% of separation efficacy to the media. In some embodiments, cleaning, sanitizing and/or regenerating includes cleaning, sanitizing and/or regenerating at least a portion of the media, e.g., at least 1%, 5%, 10%, 25%, 50% . . . 100% of the media.

As used herein, the term “microbiological contaminants” refers to any microbial contaminant. Examples of such microbiological contaminants include, but are not limited to fungi, bacteria, viruses, protista, mildew, molds, mold spores, Tuburculosis, Coronavirus, HIV, Hepatitis A, Rotovirus, Feline calici, Poliovirus, Pseudomonas aeruginosa, Staphylococcus aureus, Salmonella choleraesuis, Enterococcus faecalis, Klebsiella pneumoniae, E. coli, Salmonella typhimurium, T. mentag, Penicillin digitatum, Stachybotrys Chartarum, Aspergillus niger, Absidia sp., Acrodontium salmoneum, Aspergillus candidus, smallpox, legionella and/or anthrax.

In some embodiments, the chlorine dioxide used herein may be generated using a reactant and an initiating agent. As used herein “reactant” refers to a reactant or a mixture of reactants that generate gas in the presence of an initiating agent. Such reactants include, but are not limited to metal chlorites (e.g., sodium chlorite), and acids (e.g., citric acid). The reactant or reactants can further include additives, such as activated hydrotalcite. As used herein “initiating agent” refers to any agent that initiates the generation of gas from the reactant. For purposes of the present invention, initiating agent includes, but is not limited to gaseous or liquid water. Moisture in the atmosphere can be used as an initiating agent.

Generation of a gas, e.g., by acid activation, is well known in the art. For example, chlorine dioxide (ClO₂) is generated from sodium chlorite and an acid, such as citric acid, in the presence of moisture. Alternatively, chlorine dioxide can be produced by the reduction of a chlorate, e.g., sodium chlorate or potassium chlorate, in the presence of an acid, e.g., oxalic acid. Another example of generation of a gas by acid activation is the activation of a sulfite, e.g., sodium bisulfite or potassium bisulfite, with an acid, e.g., fumaric acid and/or potassium bitartrate, in the presence of moisture to form sulfur dioxide. Yet another example is the acid activation of a carbonate, e.g., calcium carbonate with an acid, e.g., citric acid, to form carbon dioxide.

In some embodiments, the present invention features uses of a pure chlorine dioxide solution. The term “pure solution” is meant to embody a chlorine dioxide solution in which residues such as chlorite, chlorate and/or acid reactants that lower the pH are significantly reduced as compared to conventional solutions. Conventional solutions include, but are not limited to solutions made with generators and/or acidified chlorite solutions, tablets or powders containing reactants with zeolites or clays. For example, the pure solution can include chlorite at a level of less than about 10 ppm, less than about 5 ppm, less than about 1 ppm. In another embodiment, the solution includes chlorate at a level of less than about 10 ppm, less than about 5 ppm, less than about 1 ppm. All values between these values are meant to be encompassed herein.

In some embodiments, the pure chlorine dioxide solution has a pH of between about 3 and about 10. For example, the pure chlorine dioxide solution can have a pH of 3, 4, 5, 6, 7, 8, 9, or 10. All values between these values and ranges are meant to be encompassed herein. Without wishing to be bound by any particular theory, it is believed that, because the chlorine dioxide, e.g., chlorine dioxide in solution, is stable at a wide pH range, the system can be adjusted based upon the desired pH of the chromatography media and/or the molecule(s) of interest which have been separated. That is, the methods of the present invention may be carried out at any desired pH between about 3 and about 10. In some embodiments, the pH is chosen such that the oxidation potential of the chlorine dioxide is optimized. In some embodiments, the pH of the solution is such that the chlorine dioxide remains stable and intact. In some embodiments, the pH of the solution has little or no effect on the ability of the chlorine dioxide to regenerate, clean or sanitize the chromatography media. The chlorine dioxide solutions can be buffered aqueous solutions or unbuffered aqueous solutions. The aqueous solutions can be gaseous or liquid, or even in gel form. When in gaseous form, the solution can be a ClO₂ gas present over an aqueous solution such as a mist, vapor or fog.

Without wishing to be bound by any particular theory, it is believed that high residual levels of chlorate, chlorite and other species may have significant deleterious effects on cleaning, sanitizing or regenerating chromatography media. For example, it may be difficult or impossible to remove such species from the chromatography media, once exposed.

In some embodiments, the chlorine dioxide is a substantially pure chlorine dioxide solution. The amount of chlorine dioxide in solution can range from about 0.5 ppm to about 5000 ppm. In some embodiments, the chlorine dioxide solution at least about a 5 ppm, 10 ppm, 15 ppm, 20 ppm, 25 ppm, 30 ppm, 35 ppm, 40 ppm, 45 ppm, 50 ppm, 60 ppm, 70 ppm, 80 ppm, 90 ppm, 100 ppm, 125 ppm, 150 ppm, 175 ppm, 200 ppm, 250 ppm, 500 ppm, 1000 ppm, 5000 ppm solution. Any values and/or ranges between the values and/or ranges recited herein are meant to be encompassed by the present invention, e.g., a 38 ppm solution, a 96 ppm solution, and or a solution with a range of about 28 ppm to about 132 ppm. The chlorine dioxide solution can be a buffered aqueous solution or an unbuffered aqueous solution. If a buffered solution, the chlorine dioxide solution can be made with buffers known in the art to produce the desired pH, e.g., Tris buffer.

The methods of the present invention may be carried out at any desired temperature. In some embodiments, the present invention is carried out at a temperature of between about 1° C. and about 40° C. In some embodiments, the method is carried out at about room temperature.

In some embodiments, the time needed to clean, sanitize and/or regenerate the chromatography media using chlorine dioxide is less than conventional methods. For example, the time needed to clean, sanitize and/or regenerate chromatography media may be about 10%, 25%, 50%, 75%, 100% . . . 2×, 5×, 10× quicker than conventional methods. The time needed to regenerate the chromatography media can be about 10 hours, about 5 hours, about 2 hours, about 60 minutes, about 30 minutes, about 15 minutes, about 5 minutes, or less. Without wishing to be bound by any particular theory, it is believed that a significant amount of time can be saved in the process of cleaning, sanitizing and/or regenerating chromatography media using chlorine dioxide because the chromatography media can remain in its original vessel. Vessels for chromatography include but are not limited to columns and plates. Thus, in another embodiment, the methods of the present invention can be carried out without removing the chromatography media from its vessel. The vessel for the chromatography media, e.g., a column, can be part of a larger apparatus, such as an HPLC or other analyzer. In some embodiment, the method is carried out in situ.

The volume of solution used can vary depending, e.g., upon the volume of the chromatography media used and/or the concentration of the chlorine dioxide solution. For example, a 100 ml column may need a larger volume of chlorine dioxide than a 1 ml column. Additionally, it may be desirable to use a smaller volume of a 100 ppm solution of chlorine dioxide rather than a larger volume of a 10 ppm chlorine dioxide solution. The skilled artisan can determine, with routine experimentation, the volume necessary based upon, for example, the above criteria as well as the supplies and/or chlorine dioxide solutions available.

In some embodiments, chlorine dioxide can selectively exhibit sterilizing, disinfecting or sanitizing properties as desired, for example by varying the original concentration of the chlorine dioxide solution and/or by varying the exposure time of the contaminated item to the solution.

For the purposes of clarity, it shall be understood that the methods of the present invention can be carried out using any chlorine dioxide, including aqueous solutions, gaseous chlorine dioxide, pure solutions, and/or any commercially available chlorine dioxide solutions.

The methods of the present invention can include washing the chromatography media with the chlorine dioxide solution. In some embodiments, the chromatography media is contacted in a single wash with the chlorine dioxide. In such embodiments, the single wash may be effective enough to regenerate, clean and/or sanitize at least the desired portion of the chromatography media, e.g., at least 10%, 20%, 30%, 40%, 50% . . . 100% of the media. In other embodiments, the chromatography media is contacted in two or more washes with the chlorine dioxide. In some embodiments, the second, third, fourth, etc. wash is more effective, e.g., cleans, sanitizes and/or regenerates better, than the previous washes. In some embodiments, the second, third, fourth, etc. wash is less effective than the previous washes. The present invention is meant to encompass any number of washes, using any volume and concentration of chlorine dioxide described herein. For example a first wash may be 2 ml of 10 ppm ClO₂, a second wash may be 1 ml 50 ppm ClO₂, and a third wash may be 10 ml 25 ppm ClO₂. All washes together may clean, sanitize and/or regenerate the chromatography media. Additionally or alternatively, at least one of the washes is capable of cleaning, sanitizing and/or regenerating the media. In some embodiments, the volume of the washes is at least equal to the volume of the column.

The method can include spraying the chlorine dioxide into a space or a vessel that includes the chromatography media. The spray can be, for example, a 60% to 70% relative humidity mist containing chlorine dioxide. The sprayer can include a variety of spray systems such as high volume sprayers, low volume sprayers, aerosol sprayers, thermal pulse-jet foggers, mechanical aerosol generators, cold foggers, air assisted rotary mist applicators, nebulizers, atomizers, or others known in the art. The method may further include maintaining a desired amount of chlorine dioxide in the atmosphere disposed about the chromatography media, e.g., by sealing the vessel, so as to maintain contact between the chlorine dioxide and the chromatography media for a desired length of time.

This cleaning, sanitization and/or regeneration could be performed in the manufacture of the media, at installation, during maintenance and may be performed, as described herein, in situ without disturbing the media. That is, where the media is enclosed in a vessel used in the separation process (a column, for example) it may be beneficial to be able to clean, sanitize and/or regenerate the media in place, e.g., resulting in a less costly process. Additionally, the chlorine dioxide can be generated at the point of use.

The methods of the present invention are useful in cleaning, sanitizing and/or regenerating any chromatography media known in the art. The chromatography media can be partially spent chromatography media, completely spent chromatography media or unused/regenerated chromatography media. The chromatography media includes, but is not limited to ion exchange resins, affinity resins, Protein A resins, diatomaceous earth, silica gel, silica, silica-based support systems, agaroses and/or sepharoses. The chromatography media may also include any affinity ligands known in the art. For example, the chromatography media can include sulfopropyl (S) ligands, quaternary amino (Q) ligands, carboxymethyl (CM) ligands, and/or diethylaminoethyl (DEAE) ligands. In some embodiments, the chromatography media is Q sepharose, SP sepharose, DEAE sepharose and/or CM sepharose.

The chromatography media may have been exposed to any chemicals, proteins, solvents, or other items used in conventional chromatography prior to the cleaning, sanitizing and/or regenerating process. Thus, the methods of the present invention, in some embodiments, are effective at cleaning, sanitizing and/or regenerating chromatography media which has been exposed to any chemicals, proteins, solvents, or other items used in conventional chromatography. The chromatography media may have been exposed to proteins, for example, cation exchange (CEX) or anion exchange (AEX) proteins. Proteins which may be exposed to chromatography media include, but are not limited to conalbumin, ovalbumin, lipoxidase, β-lactoglobulin, RNase A and chymotrypsinogen. In some embodiments, the proteins exposed to the chromatography media are known to interact, e.g., weakly or strongly, with any affinity ligands present in the media.

The methods of the present invention may allow the effective lifetime of the chromatography media to be increased in comparison to conventional regeneration, cleaning and/or sanitization methods, e.g., by at least about 20%, 40%, 60%, 80%, 100% . . . 1000%. For example, the methods of the present invention can allow the chromatography media to be used at least 2, 3, 4, 5, 10, 25, 50, 75, 100 or more times. The addition of even 1 or 2 separation cycles to the life of the chromatography media using the methods of the present invention would provide an advantage, for example, a cost-saving or time-saving advantage.

In some embodiments, the chlorine dioxide does not have an adverse effect on the function of the chromatography media. For example, the chlorine dioxide may not chlorinate the chromatography media. Additionally, in some cases, it may be beneficial that the chromatography media parameters be unaffected or substantially unaffected by the chlorine dioxide. For example, the compressibility, permeability and/or pore volume may be unaffected or substantially unaffected by the chlorine dioxide solution. In some cases, it may be beneficial that at least a portion of the affinity ligands on a chromatography media remain intact. This may be advantageous in that the affinity ligands would maintain at least a portion of their ability to interact with a target protein. In other cases, it may be advantageous that the activity, functionality or functional capacity of a chromatography media is increased during at least a portion of the method. For example, chromatography media which can no longer separate due to the saturation of its affinity ligands may be regenerated using the processes of the present invention, such that their ability to separate is at least partially regained.

The methods of the present invention can also include the step of removing any residual chlorine dioxide from the chromatography media. That is, in some cases, it will be beneficial for no chlorine dioxide to remain in the chromatography media once cleaning, sanitizing and/or regenerating has been completed. Residual chlorine dioxide can be removed from the chromatography media under vacuum. Additionally or alternatively, the residual chlorine dioxide can be removed by flushing the chromatography media with water, e.g., DI water, or buffer. For example, 1, 2, 5, 10, 20 or more volumes of water or buffer may be used to flush the media. Indeed, the methods of the present invention can provide a means to clean, sanitize and/or disinfect chromatography media, without leaving residual byproducts from the chlorine dioxide.

The methods of the present invention can further include storing the chromatography media in the chlorine dioxide. This may be useful in ensuring that the chromatography media remains clean and sanitized over an extended period of time. The methods of the present invention can be used to remediate microbiological contaminants. As used herein, the term “remediation” refers generally to decreasing the concentration of at least one microbiological contaminant in a sample. For example, the concentration of a contaminant can be decreased by degrading the contaminant. The microbiological contaminants can be any of those described herein. The cleaning, sanitizing and/or regenerating methods may decrease the number of colony forming units to less than about 1×10⁵, 1×10⁴, 1×10³, 1×10², 1×10 or less. Additionally or alternatively, there is a greater than 25%, 50%, 75%, 90%, or 99% reduction in viable cell count subsequent to contacting the chromatography media with the chlorine dioxide. Without wishing to be bound by any particular theory, it is also believed that microbes do not build up a resistance to the chlorine dioxide. Thus, the chlorine dioxide may generally not lose its effectiveness at remediating microbiological contaminants. In some embodiments, the chlorine dioxide destroys biofilm matrix.

The present invention further contemplates use of other gases. The gas should be capable of sanitizing, disinfecting or regenerating chromatography media as necessary. Other gases include, but are not limited to, sulfur dioxide, nitrogen dioxide, nitric oxide, nitrous oxide, carbon dioxide, hydrogen sulfide, hydrocyanic acid and dichlorine monoxide. It also should be understood that the methods of the present invention also are readily applicable to cleaning, sanitizing and/or regeneration of chromatography media using more than one gas at one time. For example, both chlorine dioxide and sulfur dioxide may be used to clean chromatography media. Additional gasses may also be used in combination with chlorine dioxide, e.g., N₂ and O₂.

The present invention also contemplates the use of kits. In a particular embodiment according to the invention, a kit includes a chlorine dioxide solution or a reactant and/or device for the production of chlorine dioxide solution. Optionally, the kit can include instructions for use of chlorine dioxide solutions for cleaning, sanitizing or regenerating chromatography media (e.g., a chart showing the amount and concentration of chlorine dioxide to be used for a specific stationary phase, affinity ligand or protein).

The following examples are expected to be illustrative of the invention and in no way limit the scope of the invention.

### EXEMPLIFICATION

### Example 1

Batch Experiments

The potential of different concentrations of chlorine dioxide for regeneration of chromatography media was screened, along with a comparison to 1.5 M NaOH and an investigation of the effects of chlorine dioxide solution on stationary phase.

Sepharose (cross-linked agarose) and Source (Polystyrene/divinylbenzene) were used as the stationary phase of the chromatography media with SP (Sulfopropyl) and Q (Quaternary amino) ligands & CM (Carboxymethyl) and DEAE (Diethylaminoethyl) as ligands. The stationary phase and ligands were chosen solely as a representative of commonly commercially used chromatography media. Generally, cation exchange proteins (RNase A, α-chymotrypsinogen at pH 6) and anion exchange proteins (conalbumin, ovalbumin, lipoxidase, β-lactoglobulin at pH 7.5) were loaded at 3 mg/ml or 6 mg/ml.

The stationary phase is first equilibrated with 3 mg/ml protein (1:12 v/v) overnight. After equilibration, excess protein in supernatant is removed leaving an approximate 50% slurry, which is mixed well. 50 μL of the slurry is pipetted into each well of a 96 well plate. The stationary phase is then filtered, leaving approximately 25 μL of the stationary phase in each well. 300 μL of chlorine dioxide (or NaOH) at varying concentrations as provided below is added to each well. The slurry is then equilibrated for 1 hour and the supernatant is removed by filtration. The regeneration ability of the chlorine dioxide solutions were then measured as overall percent regeneration of protein binding capacity.

For first, second and third regeneration screens, 3 mg/ml ribonuclease A and α-chymotrypsinogen (CEX) and conalbumin and lipoxidase or ovalbumin (AEX) were screened. Also, 5 ppm, 10 ppm, 15 ppm, 20 ppm, 25 ppm, 50 ppm, 100 ppm chlorine dioxide solutions and 1.5 M NaOH were used as regeneration solutions at binding conditions of 50 mM Phosphate buffer at pH 6 for CEX and 50 mM Tris buffer at pH 7.5 for AEX. For the fourth regeneration screen, 6 mg/ml ribonuclease A and α-chymotrypsinogen (CEX) and conalbumin, ovalbumin and β-lactoglobulin A (AEX) were screened using the same regeneration solutions and binding conditions.

First Regeneration Screen: Stationary Phase: SP and Q Sepharose (HP)

TABLE 1
Overall percent regeneration

| | | α-chymo- | | |
|---|---|---|---|---|
| | RNase A | trypsinogen | Conalbumin | Lipoxidase |
| 5 ppm | 19.70 | 0.17 | 7.66 | 14.68 |
| 10 ppm | 14.66 | 0.87 | 0.00 | 5.01 |
| 15 ppm | 13.88 | 3.14 | 12.81 | 1.55 |
| 20 ppm | 7.68 | 2.46 | 23.75 | 9.84 |
| 25 ppm | 5.71 | 1.47 | 34.66 | 4.16 |
| 50 ppm | 5.99 | 0.78 | 73.56 | 17.26 |
| 100 ppm | 5.86 | 2.91 | 77.00 | 34.76 |
| NaOH (1.5M) | 100.03 | 91.97 | 86.56 | 74.97 |

Repetition of the experiment for RNase A and conalbumin provides similar results. It can be seen from the percent regeneration in these cases that chlorine dioxide seems to be particularly effective for AEX proteins. It is believed, however, that chlorine dioxide would be effective for CEX proteins under different reaction conditions. From an experiment carried out as above, except using two washes, it was demonstrated that, in general, more protein was removed in the second wash.

Second Regeneration Screen: Stationary Phase: Source 15 S and 15 Q

TABLE 2
Overall percent regeneration

| | | α-chymo- | | |
|---|---|---|---|---|
| | RNase A | trypsinogen | Conalbumin | Ovalbumin |
| 5 ppm | 11.03 | 0.95 | 1.96 | 3.96 |
| 10 ppm | 11.32 | 0.62 | 0.00 | 1.73 |
| 15 ppm | 7.16 | 2.25 | 0.00 | 1.66 |
| 20 ppm | 7.19 | 1.62 | 4.51 | 0.00 |
| 25 ppm | 6.68 | 1.35 | 18.62 | 0.00 |
| 50 ppm | 8.76 | 2.41 | 87.40 | 26.74 |
| 100 ppm | 11.48 | 4.37 | 99.09 | 69.47 |
| NaOH (1.5M) | 133.74 | 111.24 | 137.82 | 86.87 |

Repetition of the experiment for RNase A and conalbumin provides similar results. Again, it can be seen from the percent regeneration in these cases that chlorine dioxide seems to be particularly effective for AEX proteins.

Third Regeneration Screen: Stationary Phase: Sepharose (Fast Flow) CM & DEAE

TABLE 3
Overall percent regeneration

| | | α-chymo- | | |
|---|---|---|---|---|
| | RNase A | trypsinogen | Conalbumin | Ovalbumin |
| 5 ppm | 1.97 | 0.43 | 14.29 | 21.47 |
| 10 ppm | 3.67 | 0.83 | 0.00 | 9.61 |
| 15 ppm | 2.21 | 3.14 | 0.00 | 7.55 |
| 20 ppm | 2.13 | 3.81 | 0.00 | 12.80 |
| 25 ppm | 0.96 | 3.66 | 0.87 | 10.42 |
| 50 ppm | 3.01 | 6.36 | 11.49 | 18.82 |
| 100 ppm | 8.89 | 7.91 | 88.47 | 78.52 |
| NaOH (1.5M) | 87.54 | 69.02 | 143.42 | 137.84 |

Again, it can be seen from the percent regeneration in these cases that chlorine dioxide, particularly 100 ppm chlorine dioxide, seems to be particularly effective for AEX proteins.

Fourth Regeneration Screen: Stationary Phase: SP & Q Sepharose (HP)

TABLE 4
Overall percent regeneration

| | RNase A | α-chymotrypsinogen | Conalbumin | Ovalbumin | β-lactoglobulin |
|---|---|---|---|---|---|
| 5 ppm | 0.00 | 1.67 | 2.10 | 0.28 | 0.20 |
| 10 ppm | 0.00 | 1.53 | 6.08 | 0.00 | 0.00 |
| 15 ppm | 0.00 | 2.27 | 2.09 | 0.00 | 0.00 |
| 20 ppm | 0.00 | 2.84 | 9.47 | 0.00 | 0.00 |
| 25 ppm | 0.00 | 1.64 | 20.49 | 0.00 | 0.00 |
| 50 ppm | 0.00 | 2.08 | 74.09 | 22.01 | 7.64 |
| 100 ppm | 0.49 | 2.54 | 81.42 | 136.52 | 46.26 |
| NaOH (1.5M) | 122.58 | 96.64 | 92.90 | 89.75 | 85.49 |

The results obtained from the fourth regeneration screen indicate that protein regeneration is relatively independent of the initial protein loading. (The trends at 6 mg/ml are similar to that at 3 mg/ml.) Again, regeneration is generally more effective for AEX proteins than for CEX proteins. Additionally, it was demonstrated that β-lactoglobulin, another AEX protein is also effectively regenerated from the column, although to a lower extent than conalbumin or ovalbumin.

Thus, it is demonstrated from the four regeneration screens that the performance of the chlorine dioxide solution is generally independent of backbone chemistry (Sepharose VS Source), ligand chemistry (SP &Q VS CM & DEAE) and/or protein loading (3 mg/ml VS 6 mg/ml). Additionally, it was demonstrated that performance was somewhat protein specific for AEX proteins, with conalbumin and ovalbumin at greater than 70% regeneration and lipoxidase and β-lactoglobulin at approximately 40-50% regeneration. Furthermore, for AEX proteins, more protein was generally removed in second wash, and thus improvement may be possible by increasing chlorine dioxide concentration or providing more washes.

Calibration Check

The absorbance due to chlorine dioxide solution was checked against the absorbance due to a buffer solution, because the chlorine dioxide may interfere with protein absorbance. It is noted that this effect is unlikely to be significant since most chlorine dioxide (gas) would be removed during vacuum filtration. The calibration check demonstrates that, for 3 mg/ml conalbumin protein prepared in NaOH, 100 ppm chlorine dioxide and buffer, the error was less than 15%. This is at highest protein concentration and discrepancy is smaller at lower protein concentrations. Additionally, the curve for protein in chlorine dioxide is higher than curve for protein in buffer. Thus, in addition to the trends identified herein not being affected, the actual regeneration results for chlorine dioxide (50 ppm and 100 ppm) may be 10%-15% higher than predicted using calibration curve in buffer.

pH Shift Effects

Unbuffered chlorine dioxide (in DI) and buffered chlorine dioxide (in 50 mM Tris pH˜7.5) were analyzed based on amount of protein removed (mg) in a high throughput batch format. Precise measurement of pH was not possible due to the high resistivity of the water and the purity of the solution. The comparison study was carried out focusing on the amount of protein removed from the stationary phase during regeneration (in mg). For the unbuffered system, the total amount of protein removed after 2 washes for 50 ppm and 100 ppm chlorine dioxide were comparable (approximately 0.15 mg). For the unbuffered system, however, little or no protein removal from the stationary phase was detected. Thus, it is believed that pH has a role to play in the regeneration of protein from AEX systems.

### Example 2

Effect of Chlorine Dioxide on Stationary Phase

The stationary phase (with no protein) was treated with buffer or 300 μL of chlorine dioxide for 1 hour. The chlorine dioxide or buffer was then removed by vacuum filtration, and the stationary phase was washed 3 times with 300 μL buffer. 150 μL protein (RNase A and conalbumin, 3 mg/ml) was added to the stationary phase and left to equilibrate overnight. The supernatant was removed and absorbance measured to determine the percent of protein bound to the stationary phase. The amount of protein bound to the stationary phase treated with chlorine dioxide was then compared to the stationary phase treated with the control buffer. Results, in percentage protein bound after treatment with chlorine dioxide, are shown in Tables 5-7.

TABLE 5
Stationary phase: Sepharose (HP)//SP & Q

| | |
|---|---|
| RNase A | Conalbumin |

| | | | | |
|---|---|---|---|---|
| Buffer | 93.07 | 94.12 | 64.77 | 61.93 |
| 5 ppm | 92.82 | 92.95 | 71.83 | 59.64 |
| 25 ppm | 94.21 | 91.62 | 71.02 | 68.05 |
| 50 ppm | 96.30 | 98.15 | 72.09 | 71.61 |
| 100 ppm | 98.35 | 97.03 | 73.72 | 72.77 |
| No stationary phase | 0.00 | 0.00 | 0.00 | 0.00 |

TABLE 6
Stationary phase: Source 15 S and 15 Q

| | |
|---|---|
| RNase A | Conalbumin |

| | | | | |
|---|---|---|---|---|
| Buffer | 91.30 | 91.13 | 49.49 | 54.35 |
| 5 ppm | 91.17 | 91.62 | 54.53 | 52.55 |
| 25 ppm | 92.58 | 89.71 | 51.84 | 56.73 |
| 50 ppm | 90.02 | 91.65 | 57.76 | 58.52 |
| 100 ppm | 90.08 | 91.13 | 52.12 | 47.45 |
| No stationary phase | 0.00 | 0.00 | 0.00 | 0.00 |

TABLE 7
Stationary phase: Sepharose (Fast Flow) CM & DEAE

| | |
|---|---|
| RNase A | Conalbumin |

| | | | | |
|---|---|---|---|---|
| Buffer | 87.95 | 88.19 | 64.05 | 64.05 |
| 5 ppm | 89.47 | 80.49 | 63.84 | 58.48 |
| 25 ppm | 79.18 | 86.88 | 65.12 | 62.74 |
| 50 ppm | 90.22 | 87.95 | 66.14 | 63.75 |
| 100 ppm | 86.31 | 90.10 | 69.74 | 65.55 |
| No stationary phase | 0.00 | 0.00 | 0.00 | 0.00 |

For the stationary phases studied in Example 1: SP and Q Sepharose (HP), Source 15 S and 15 Q, and Sepharose (Fast Flow) CM & DEAE, the chlorine dioxide solution had no significant effect on stationary phase in comparison to the control buffer. This is advantageous because the stationary phase should retain at least a portion of its functionality as a chromatography media.

### Example 3

Column Experiments

Preliminary column experiments were undertaken to verify results from batch experiments and to determine column volumes required for regeneration of chromatography media.

Frontal Experiment

In order to establish breakthrough time, conalbumin protein (3 mg/ml) was passed through a self-packed Source 15Q column (1 ml). It was demonstrated, in an analysis of absorbance versus time, that the breakthrough time for 3 mg/ml conalbumin protein in the self-packed Source 15Q column is 12-14 minutes.

Column Experiment 1

A solution of 100 ppm chlorine dioxide was initially prepared. (The pH was measured at 4.5.) A Source 15Q chromatography column (self-packed) with a volume of 1 ml was employed. To ensure that the column was fully regenerated before the experiment, it was rinsed with 15 column volumes of a 2M sodium chloride solution. For equilibration, 15 column volumes of 20 mM Tris buffer at pH 7.5 were employed. The first frontal experiment was carried out by feeding the column with 3 mg/ml Conalbumin protein at a flowrate of 0.2 ml/min and the breakthrough time was recorded. The column was then regenerated using an additional 15 column volumes of 2M sodium chloride, followed by re-equilibration with 15 column volumes of 20 mM Tris buffer at pH 7.5. A second frontal was carried out to confirm the breakthrough time. The column was then regenerated with 15 column volumes of the 100 ppm chlorine dioxide solution. The column was rinsed with 15 column volumes to DI water to remove residual chlorine dioxide solution and a third frontal was carried out.

The frontal experiments were done to assess column capacity before and after regeneration with chlorine dioxide. From a plot of absorbance versus time, it was demonstrated that the breakthrough time from Frontal 3 (after regeneration with chlorine dioxide) was almost identical to the breakthrough times from Frontals 1 & 2. Without wishing to be bound by any particular theory, these results are believed to be important because the changing the ionics of the chromatography media can have a dramatic effect on the breakthrough time. These results obtained upon treatment with chlorine dioxide verify that complete regeneration can be achieved using chlorine dioxide for an AEX column and conalbumin protein with little effect on the ionics of the chromatography media.

Column Experiment 2

From the high throughput batch experiment comparing the performance of buffered and unbuffered chlorine dioxide solutions, it was concluded that pH effect had a role in the regeneration behaviors observed. To further investigate whether it is pH effect alone or a combination of pH and chlorine dioxide effect, this column experiment focuses on evaluating regeneration of AEX column using a buffer with a pH of 4.5, (in the absence of chlorine dioxide).

25 mM Citrate buffer pH 4.5 was prepared and the same experimental format was adopted as column experiment 1. A Source 15Q chromatography column (self-packed) with a volume of 1 ml was employed and conalbumin protein with a concentration of 3 mg/ml was again used as the test protein. Initially a frontal experiment was carried out to check the breakthrough time of the protein. The column was then regenerated using 15 column volumes of citrate buffer and a second frontal was carried out.

The frontals before and after regeneration with citrate buffer pH 4.5 indicate that the breakthrough time in Frontal 2 (after regeneration with citrate buffer) is lower than the breakthrough time in Frontal 1 (clean column). This indicates some loss of column capacity attributed to remaining protein adsorbed onto the stationary phase. From the results, it is believed that the mechanism behind regeneration for AEX proteins may be a combination of pH effects and the effect of chlorine dioxide itself.

Prospective—Regeneration of Columns in Situ

A saturated column with any one of the proteins listed herein (3 mg/ml) and any one of the chromatography media (1 ml) will be prepared generally as described above. The column will then be regenerated using chlorine dioxide solutions of varying concentrations and 1.5 M NaOH. It is expected that the chlorine dioxide will be able to regenerate the column at least as effectively as in the batch and column experiments above. Subsequent to regeneration, the new breakthrough time will be checked by carrying out another frontal experiment to compare with the original breakthrough time.

Equivalents

Those skilled in the art will recognize, or be able to ascertain using no more than routine experimentation, many equivalents to the specific embodiments of the invention described herein. Such equivalents are intended to be encompassed by the following claims.

1. A method for treating chromatography media comprising: contacting chromatography media with chlorine dioxide, wherein the media is at least partially treated.

2. The method of claim 1, wherein the media is at least partially cleaned or sanitized.

3. The method of claim 1, wherein the media is at least partially regenerated.

4. A method for regenerating, cleaning or sanitizing chromatography media in situ comprising: contacting chromatography media with chlorine dioxide without removing the chromatography media from its vessel, wherein the media is at least partially regenerated, cleaned or sanitized.

5. The method of any one of the preceding claims, wherein the chlorine dioxide is a substantially pure chlorine dioxide solution.

6. The method of any one of the preceding claims, wherein the chlorine dioxide solution is at least about a 5 ppm, 25 ppm or 100 ppm chlorine dioxide solution.

7-8. (canceled)

9. The method of any one of the preceding claims, wherein the chlorine dioxide solution is an unbuffered aqueous solution.

10. The method of any one of the preceding claims, wherein the chromatography media is at least one media selected from the group consisting of an ion exchange resin, an affinity resin, a Protein A resin, a diatomaceous earth, a silica gel, a silica, a silica-based support system, agarose, sepharose, controlled pore glass and size exclusion media.

11. The method of any one of the preceding claims, wherein the chromatography media is at least one media selected from the group consisting of an affinity resin, a silica-based support system and a sepharose.

12. The method of any one of the preceding claims, wherein the chromatography media has been exposed to cation exchange (CEX) or anion exchange (AEX) proteins or both.

13. The method of any one of the preceding claims, wherein the pH of the chlorine dioxide in solution is between about 3.0 and about 10.0.

14. The method of any one of the preceding claims, wherein the pH of the solution is such that chlorine dioxide remains stable and intact.

15. The method of any one of the preceding claims, wherein the pH has little or no impact on the ability of the chlorine dioxide to regenerate, clean or sanitize the chromatography media.

16. The method of any one of the preceding claims, wherein the contacting step comprises a single wash with the chlorine dioxide.

17. The method of any one of the preceding claims, wherein the contacting step comprises two or more washes with the chlorine dioxide.

18. The method of any one of the preceding claims, wherein the method allows the effective lifetime of the chromatography media to be increased by at least 20% in comparison to conventional regeneration, cleaning or sanitization methods.

19. The method of any one of the preceding claims, wherein the method allows the chromatography media to be used at least two, five or twenty times.

20-21. (canceled)

22. The method of any one of the preceding claims, wherein the method is carried out without removing the chromatography media from its vessel.

23. The method of any one of the preceding claims, wherein the method is carried out in situ.

24. The method of any one of the preceding claims, wherein the chlorine dioxide does not have an adverse effect on the function of the chromatography media.

25. The method of any one of the preceding claims, wherein the chromatography media parameters are substantially unaffected by the chlorine dioxide.

26. The method of any one of the preceding claims, wherein any affinity ligands on the chromatography media remain intact during the method.

27. The method of any one of the preceding claims, wherein the functional capacity of the chromatography media is increased during at least a portion of the method.

28. The method of any one of the preceding claims, wherein the chlorine dioxide does not chlorinate the chromatography media.

29. The method of any one of the preceding claims, further comprising removing any residual chlorine dioxide from the chromatography media.

30. The method of claim 29, wherein the removal of residual chlorine dioxide is done under vacuum.

31. The method of any one of the preceding claims, further comprising flushing with 1 to 2 volumes of water or buffer.

32. The method of any one of the preceding claims, further comprising storing the chromatography media in the chlorine dioxide.

33. The method of any one of the preceding claims, wherein the method is carried out at about room temperature.

34. The method of any one of the preceding claims, wherein the chlorine dioxide is generated at the point of use.

35. The method of any one of the preceding claims, wherein the chlorine dioxide remediates microbiological contaminants.

36. The method of claim 35, wherein the microbiological contaminants comprise at least one contaminant selected from the group consisting of fungi, bacteria, viruses, protista, mildew, molds, mold spores, Tuburculosis, Coronavirus, HIV, Hepatitis A, Rotovirus, Feline calici, Poliovirus, Pseudomonas aeruginosa, Staphylococcus aureus, Salmonella choleraesuis, Enterococcus faecalis, Klebsiella pneumoniae, E. coli, Salmonella typhimurium, T. mentag, Penicillin digitatum, Stachybotrys Chartarum, Aspergillus niger, Absidia sp., Acrodontium salmoneum, Aspergillus candidus, smallpox, legionella and anthrax.

37. The method of claim 35, wherein the amount of colony forming units subsequent to contacting the chromatography media with the chlorine dioxide is less than about 10.

38. The method of claim 35, wherein there is a greater than 99% reduction in viable cell count subsequent to contacting the chromatography media with the chlorine dioxide.

39. The method of claim 35, wherein any microbes do not build up a resistance to the chlorine dioxide.

40. The method of claim 35, wherein chlorine dioxide destroys any biofilm matrix.

41. The method of any one of the preceding claims, further comprising contacting the chromatography media to at least one gas selected from the group consisting of N₂, O₂, sulfur dioxide, nitrogen dioxide, nitric oxide, nitrous oxide, carbon dioxide, hydrogen sulfide, hydrocyanic acid and dichlorine monoxide.

42. The method of any of the preceding claims, wherein the chromatography media is sterilized.

43. A kit comprising: chlorine dioxide and instructions for using the chlorine dioxide to regenerate, clean, or sanitize chromatography media.
